(19) United States
(12) Patent Application Publication
(10) Pub. No.: US 2007/0191456 A1
(43) Pub. Date: Aug. 16, 2007

(54) Novel process for the preparation of 1-(9h-carbazol-4-yloxy)-3-[[2-(-methoxyphenoxy)-ethyl] amino]-propan-2-ol

(76) Inventors: Venkatasubramanian Radhakrishnan Tarur, Maharashtra (IN); Dhananjay Govind Sathe, Maharashtra (IN); Swapnil Jayant Kulkarni, Maharashtra (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/568,732

(22) PCT Filed: May 3, 2005
(86) PCT No.: PCT/IN05/00139

§ 371 Date: Dec. 27, 2006

(30) Foreign Application Priority Data
May 7, 2004 (IN) 479/MUM/2004

(51) Int. Cl. A61K 31/403; C07D 209/82

(52) U.S. Cl. 514/411; 548/444

(57) ABSTRACT

The present invention discloses a novel process for preparation of carvedilol by using eco friendly solvents to obtain the said carvedilol in high purity. The said process comprises, reacting 4-hydroxy carbazole of formula (IV) with epichlorhydrin in presence of an organic solvent and a base at temperatures between 10° C.-30° C.; further reacting the resultant 4-(2,3-epoxypropoxy)- carbazole of formula (II) with a salt of 2-(2-methoxyphenoxy)ethylamine of formula (III), preferably hydrochloride salt in presence of a base and a hydroxylic solvent at temperatures between 30° C.-90° C.

### RELATED APPLICATIONS

This application claims priority from India National patent application serial No. 479/MUM/2004, filed on 7 May 2004.

### TECHNICAL FIELD OF INVENTION

This invention relates to a novel process for preparation of carvedilol (1) by using eco friendly solvents to obtain the said carvedilol in high purity. Carvedilol is a compound useful in the treatment of hypertension and angina.

### BACKGROUND AND PRIOR ART

Carvedilol is a nonselective β-adrenergic blocking agent, with α₁ blocking activity. Carvedilol, the first beta blocker labeled in the United States for the treatment of heart failure, has been shown to improve left ventricular ejection fraction and may reduce mortality. Carvedilol is chemically known as 1-(9H-carbazol-4yloxy)-3-[[2-(-methoxyphenoxy)-ethyl]amino]-propan-2-ol, of formula (I), is given below.

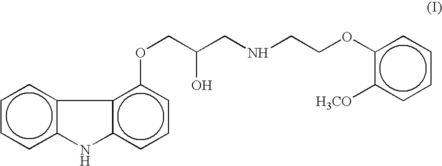

As depicted in scheme 1, the U.S. Pat. No. 4,503,067 describes preparation of (a) by reacting 4-(2,3-epoxypropoxy)-carbazole (II) with 2-(2-methoxyphenoxy)-ethylamine (III) using ethylene glycol, dimethyl ether as solvent. The reaction is reported to be carried out for 25 hours at 50° C. The crude Carvedilol (I) produced, is further crystallized.

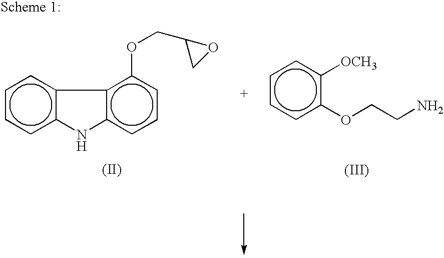

### CARVEDILOL (I)

As depicted in scheme 2 the British patent application GB 1369580 reports the preparation of 4-(2,3-epoxypropoxy)-carbazole, compound of formula (II) from 4-hydroxy carbazole, compound of formula (IV) and epichlorohydrin using aqueous sodium hydroxide in solvents like 1,4 dioxan; as shown in scheme 2

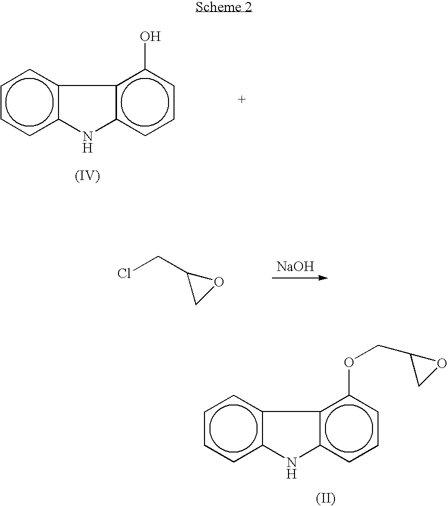

A major drawback of the process reported in U.S. Pat. No. 4,503,067 is the formation of bis-compound formed by reaction of Carvedilol (I) with one more molecule of II. The problem is partly overcome by using the N-benzyl derivative of 2-(2-methoxyphenoxy)-ethylamine (III) instead of the free amine as reported in patent EP 0918055 A₁ and Indian Patent 186587, but the N-Benzyl Carvedilol formed needs to be debenzylated using palladium catalyst. Use of palladium increases the cost of the process and also poses the hazards of handling palladium, which is pyrophoric in presence of solvent vapours. This procedure involves additional process steps, which is benzylation and subsequent debenzylation steps resulting in lower yields.

U.S. Pat. No. 6,699,997 reports the preparation of carvedilol, which involves the reaction between 2-(2-methoxy phenoxy)ethyl amine and 4-(oxiran-2-ylmethoxy)-9H-carbazole at 100° C.

To avoid the hazardous axiranyl derivatives in the process for preparation of carvedilol, which is desirable for environmental reasons, WO0187837 describes another process for preparation of carvedilol or an acid addition salt thereof, prepared by alkylating 5-chloromethyl-3-[2-(2-methoxy-phenoxy)-ethyl]-oxazolidine-2one with 4-hydroxy-carbazole for the formation of 5-(9H-carbazol-4-yloxy methyl)-3-[2-(2-methoxy-phenoxy)-ethyl}-oxazolidine-2-one or an acid addition salt thereof, which is subsequently decarboxylated.

The process reported in GB-1369580 for the preparation of the key intermediates of 4-(2,3-epoxypropoxy)-carbazole, formula (II) uses 1,4-dioxan as a solvent. 1,4-dioxan is an expensive, high boiling solvent and its removal from the product is quite tedious. The complete removal is very difficult requiring the product formed, to be extracted in solvents like dichloromethane and the dichloromethane layer washed with water to remove traces of 1,4-dioxan. The separation of two immiscible layers is quite cumbersome as aqueous and organic layers are both highly colored and hence difficult to distinguish. Moreover, effluent containing 1,4 dioxan is also posing disposal problems.

The process mentioned in U.S. Pat. No. 4,503,067, although reports preparation of Carvedilol as a single step reaction between 4-(2,3-epoxypropoxy)-carbazole, the compound of formula II with 2-(2-methoxyphenoxy)-ethylamine of formula III, it is essentially a two stage process. 2-(2-methoxyphenoxy)-ethylamine III is commercially available as its hydrochloride salt. First stage involves liberating the amine as a free base from the hydrochloride salt using alkali and extracting the amine thus liberated into organic solvents and solvents removed under reduced pressure. Second stage comprises mainly of reacting the free amine with compound of 4-(2,3-epoxypropoxy)-carbazole, formula (II) to obtain Carvedilol. Hence the process reported above is not the preferred one in a production plant since it involves use of more than one reactor. Moreover, condensation of compound of formula II with 2-(2-methoxyphenoxy)-ethylamine of formula III is carried out in solvents like ethylene glycol, dimethyl ether, which is very expensive.

Objective

An object of the present invention is to provide a process for the commercial manufacture of 1-(9 H-carbazol-4-yloxy)-3-[[2-(2-methoxyphenoxy)-ethyl]amino]-propan-2-ol, known as Carvedilol, which is simple to carryout in the plant, uses eco friendly solvents and is economical.

Another object of the invention is to provide a process for Carvedilol, which is less time consuming involving fewer steps and increases the production efficiency.

A further object of the invention is to provide a process for Carvedilol, which avoids use of hazardous reagents and use milder reaction conditions.

Another object of the invention is to provide a process for Carvedilol, which employs use of less expensive, eco friendly solvents.

### SUMMARY OF THE INVENTION

The present invention discloses a novel process for preparation of carvedilol (I) by using eco friendly solvents to obtain the said carvedilol in high purity wherein, the said process comprises, reacting 4-hydroxy carbazole of formula (IV) with epichlorhydrin in presence of an organic solvent and a base at temperatures between 10° C.-30° C.; further reacting the resultant 4-(2,3-epoxypropoxy)-carbazole of formula (II) with a salt of 2-(2-methoxyphenoxy)ethylamine of formula (III), preferably hydrochloride salt in presence of a base and a hydroxylic solvent at temperatures between 30° C.-90° C.

The preferred base is inorganic base preferably alkali metal hydroxide, more-preferably sodium hydroxide in aqueous form.

The molar equivalent of base is employed may be from 1 mole to 6 moles, preferably 1.1 molar equivalents based on 4-hydroxy carbazole moles.

The organic solvent is selected from alcohols, cyclic ethers, dipolar aprotic solvents and glycol ethers, preferably water miscible (C1-C4) alcohols but, more preferably isopropyl alcohol.

The hydroxylic solvent is water or C₁-C₄ alcohols like methyl alcohol, ethyl alcohol, isopropyl alcohol, butyl alcohol or mixtures thereof but preferably water.

The preferred temperature range is 20-30° C. in the reaction between 4-hydroxy carbazole of formula (IV) and epichlorhydrin. The preferred temperature range is 80° C.-90° C. in the reaction between the compounds of formula II and formula III.

The process disclosed in the present invention involves fewer and simpler steps, avoids use of hazardous reagents; uses milder and eco-friendly reagents and solvents.

### DETAILED DESCRIPTION OF THE INVENTION

According to the invention there is provided a process for the preparation of 1-(9H-carbazol-4-yloxy)-3-[[2-(2-methoxyphenoxy)-ethyl]amino]-propan-2-ol, (Carvedilol) of formula I, comprising of two steps:

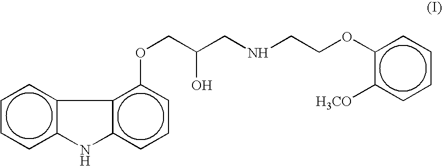

Step I: Preparation of compounds of formula II by reacting compounds of formula IV with epichlorohydrin in water miscible organic solvents containing solution of a base at temperatures ranging from 10 to 60° C. as shown in scheme 3.

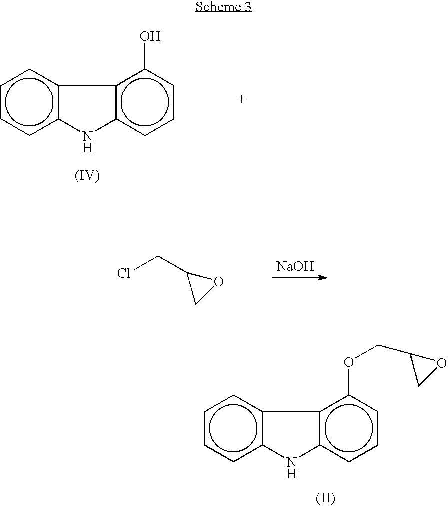

The organic solvents are selected from alcohols, cyclic ethers, dipolar aprotic solvents and glycol ethers but preferably water miscible (C₁-C₄) alcohols and more preferably Isopropyl alcohol.

Bases may be selected from organic like alkyl amines, inorganic like alkaline earth metal and alkali metal salts like sodium carbonate, sodium bicarbonate, sodium hydroxide, calcium hydroxide, barium hydroxide and the likes thereof. Solution of the base includes any one of the above bases in water or any previously mentioned water miscible solvents. The preferred base is sodium hydroxide in water.

The molar equivalent of base employed may be from 1 mole to 6 moles based on IV, preferably 1.1 molar equivalents.

The co-alkylation can be carried out at temperatures from 10° C. to 60° C. but preferably at 20-30 ° C.

The process thus involves use of eco friendly solvents like isopropyl alcohol in aqueous inorganic base like sodium hydroxide giving 4-(2,3-epoxypropoxy)-carbazole (II) in 84% yield. This process eliminates use of expensive solvents like 1,4 dioxan and obnoxious solvents like dimethyl sulphoxide. This process further has the benefit of ease of adaptability in the plant.

Step II: preparation of compounds of formula (I) by reacting compounds of 4-(2,3-epoxypropoxy)-carbazole of formula (II) with amine salts of 2-(2-methoxyphenoxy)-ethylamine of Formula (III) in solvents and in presence of base at temperatures from 30-90° C. as shown in scheme 4, where R═H, CH₂Ph.

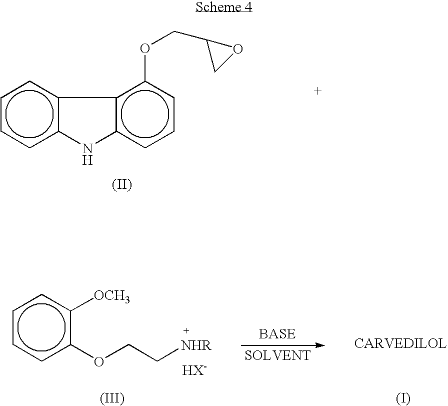

The salts of 2-(2-methoxyphenoxy)-ethylamine of Formula (III) that may be used include mineral acid salts like hydrochloride, sulphate, phosphate, or organic acids salts like tartarate, methane sulphonate, para toluene sulphonate, citric acid, malic acid, oxalic acid salts and others there of but preferably the hydrochloride salt which is cheaper and commercially available.

The solvents include hydroxylic solvents like water, alcohols, water miscible solvents like dimethyl sulphoxide, glycol ethers, acetone but preferably water, as it is the cheapest and most eco friendly solvent.

The bases that may be used for neutralizing the acid addition salts of the amine in situ may be any of the organic bases like trialkylamines, inorganic bases like alkali metal and alkaline earth salts but preferably sodium hydroxide which is very cheap and making the process commercially viable. The condensation is carried out at temperature from RT to 90° C. preferably at 80-85° C. and the reaction is over in about 30 minutes to an hour.

Thus the process of manufacturing Carvedilol by the present invention comprises, use of solvents like water and isopropyl alcohol which are cheap, freely available and eco friendly. The process of the invention involves less number of steps since commercially available 2-(2-methoxyphenoxy)-ethylamine hydrochloride can be directly condensed with 4-(2,3 epoxypropoxy) carbazole to give Carvedilol without going through an additional step of converting the amine hydrochloride to its free base before condensation with epoxypropoxy carbazole. This eliminates use of solvents which are expensive and eco unfriendly. Overall the process of making carvedilol by the present invention is a novel process, which is eco friendly and industrially viable. The process of this invention completes in lesser reaction times.

### EXAMPLE 1

### 4-(2,3-Epoxypropoxy)-carbazole (II)

200.0 g (1.09 mole) of 4-hydroxy carbazole is dissolved in 500.0 ml of isopropyl alcohol. To this solution is added dropwise an aqueous solution of sodium hydroxide made from 48 g (1.2 moles) of sodium hydroxide dissolved in 700.0 ml of water. The addition of alkali solution is done by maintaining the temperature at 23 to 28° C. After the addition of entire quantity of sodium hydroxide solution, the reaction mixture is stirred for 1 hour at the same temperature of 23 to 28° C. To this solution 236 g (2.55 moles) of epichlorhydrin is added all at once. The reaction mixture is then stirred at room temperature (30-40° C.) for 15-20 hours. During this period the reaction mixture is monitored by thin layer chromatography (TLC), till the reaction showed complete utilization of 4-hydroxy carbazole. 4-(2,3-epoxypropoxy) carbazole, thus formed, precipitated from the reaction mixture, which is filtered and the cake is washed with 150 ml of water and 150 ml of isopropyl alcohol. The product is dried at 50-55° C. for 4-5 hours.

Yield: 210.0 g. (80.4%) M.P. 133° C.

¹H NMR (200 MHZ, in CDCl₃) δ (ppm) 8.1 (bs, 1H, exchanges with d₂O), 6.8-8.3 (m 7H), 4.4-4.2 (m, 2H), 3.5 (m, 1H), 2.8-3 (m, 2H)

### EXAMPLE 2

### 1-(9H-carbazol-4-yloxy)-3-[[2-(2-methoxyphenoxy)-ethyl]amino]-propan-2-ol (Carvedilol)

Method A:

To a solution of 94.0 g (0.461 moles) of 2-(2-methoxyphenoxy)-ethylamine hydrochloride in 800.0 ml water is added sodium hydroxide (pellets or flakes) at room temperature till the pH of the solution is 9 to 9.5, when the solution became clear.

To this clear solution is added 100.0 g (0.418 moles) of 4-(2,3-epoxypropoxy)carbazole all at once. The reaction mixture is then heated at 80-85° C. for 45 to 60 minutes when it is monitored by thin layer chromatography, and the TLC showed completion of reaction. The reaction mixture is worked up by the addition of 500.0 ml of ethyl acetate and stirring the mixture for 15 min, the ethyl acetate layer is separated, dried over sodium sulphate and evaporated to dryness to obtain Carvedilol, which is recrystallized from ethyl acetate.

Yield: 78.0 g (45.99%)

M.P: 114° C. (lit M.P. 113 to 116° C., Merck index 13^{th} edition)

¹H NMR (200 MHZ, in CDCl₃) δ (ppm) 8.2 (bs,1H, exchanges with d₂O), 6.8-8.3 (m 7H) 4.2-4.0 (m 1H), 3.8 (s 3H), 3-3.2 (t 4H), 2.8 (m 4H), 1.9 (bs,1H, exchanges with d₂O)

Method B:

To a solution of 94.0 g (0.461 moles) of 2-(2-methoxyphenoxy)-ethylamine hydrochloride in 400.0 ml water and 400 ml isopropyl alcohol is added sodium hydroxide (pellets or flakes) at room temperature till the pH of the solution was 9 to 9.5, when the solution became clear.

To this clear solution is added 100.0 g (0.418 moles) of 4-(2,3-epoxypropoxy)carbazole all at once. The reaction mixture is then refluxed for 4 to 5 hours when it is monitored by thin layer chromatography, and the TLC showed completion of reaction.

The reaction mixture is worked up by the addition of 500.0 ml of ethyl acetate and stirring the mixture for 15 min, the ethyl acetate layer is separated, dried over sodium sulphate and evaporated to dryness to obtain Carvedilol, which is recrystallized from ethyl acetate.

Yield: 73.0 g (43.05%)

M.P: 114 (lit M.P. 113 to 116° C., Merck index 13^{th} edition)

¹H NMR (200 MHZ, in CDCl₃) (ppm) 8.2 (bs,1H, exchanges with d₂O), 6.8-8.3 (m 7H) 4.2-4.0 (m 1H), 3.8 (s 3H), 3-3.2 (t 4H), 2.8 (m 4H), 1.9 (bs,1H, exchanges with d₂O)

1. A process for the preparation of 1-(9 H-carbazol-4-yloxy)-3-[[2-(2-methoxyphenoxy)-ethyl]amino]-propan-2-ol, (Carvedilol) comprising:
(a) reacting 4-hydroxy carbazole of formula (IV) with epichlorhydrin
in presence of an organic solvent and a base at temperatures between 10° C.-30° C.
(b) further reacting the resultant 4-(2,3-epoxypropoxy)-carbazole of formula (II) with a salt of 2-(2-methoxyphenoxy)ethylamine of formula (III), preferably hydrochloride salt in presence of a base
and a hydroxylic solvent
at temperatures between 30° C.-90° C.

2. A process as claimed in claim 1, wherein the preferred base is inorganic base preferably alkali metal hydroxide, more preferably sodium hydroxide in aqueous form.

3. A process as claimed in claim 1(b), wherein the molar equivalent of base is employed may be from 1 mole to 6 moles, preferably 1.1 molar equivalents based on 4-hydroxy carbazole moles.

4. A process as claimed in claim 1(a), wherein the said organic solvent is selected from alcohols, cyclic ethers, dipolar aprotic solvents and glycol ethers, preferably water miscible (C1-C4) alcohols but, more preferably isopropyl alcohol.

5. A process as claimed in claim 1(b), wherein the said hydroxylic solvent is water or C₁-C₄ alcohols like methyl alcohol, ethyl alcohol, isopropyl alcohol, butyl alcohol or mixtures thereof but preferably water.

6. A process as claimed in claim 1(a), wherein the preferred temperature range is 20-30° C. in the reaction between 4-hydroxy carbazole of formula (IV) and epichlorhydrin.

7. A process as claimed in claim 1(b), where in the preferred temperature range is 80° C.-90° C. in the reaction between the compounds of formula II and formula III.

8. A process for preparation of Carvedilol as substantially described herein with reference to the foregoing examples 1 to 2.
